(19) United States
(12) Patent Application Publication
(10) Pub. No.: US 2003/0232798 A1
(43) Pub. Date: Dec. 18, 2003

(54) Adrenal enzyme inhibitors

(76) Inventor: Yadon Arad, Mountain View, CA (US)

Correspondence Address: DERGOSITS & NOAH LLP, Attn: Malcolm B. Wittenberg, Suite 1450, Four Embarcadero Center, San Francisco, CA, 94111, US

(21) Appl. No.: 10/198,659

(22) Filed: Dec. 2, 2002

Related U.S. Application Data
(62) Division of application No. 09/880,624, filed on Jun. 12, 2001, now abandoned.

(51) Int. Cl. A61K 31/573

(52) U.S. Cl. 514/179

(57) ABSTRACT

A composition and method for inhibiting adrenal enzyme synthesis in a user in order to improve glucose tolerance, reduce obesity, reduce diabetes, reduce hypertension and reduce atherosclerosis. The preferred active ingredient is trilostane which is combined with a suitable carrier which acts as an adrenal enzyme inhibitor and is thus useful for treating diabetes mellitus, hypertension, obesity and atherosclerosis.

### TECHNICAL FIELD OF THE INVENTION

The present invention involves the use of adrenal enzyme inhibitors for inhibiting increased secretion of cortisol in response to stressful stimuli such as anxiety, anger, depression and fear thus ameliorating such maladies as glucocorticoid-mediated insulin resistance, hypertension, obesity and atherosclerosis.

### BACKGROUND OF THE INVENTION

Diabetes, hypertension, obesity and cardiovascular disease are the most prevalent medical conditions in western societies and are rapidly becoming the major causes of mortality and morbidity in developing countries as well. It is estimated that hypertension affects approximately 20% of the persons in the United States and world wide. The corresponding numbers for diabetes are 10-5% of the population, 10-12% for obesity and for cardiovascular disease 10-5%. Despite major advances in treating these conditions, current treatment is inadequate.

Obesity is likewise becoming more and more prevalent in western societies as well as in developed countries and is now described as an epidemic. In particular, central or intra-abdominal fat accumulation is associated with increased risk of cardiovascular disease.

These three conditions, diabetes, hypertension and obesity, co-segregate and are associated with a variety of other metabolic abnormalities such as low concentrations of HDL cholesterol, elevated concentrations of triglycerides and small dense low density lipoprotein.

Decreased blood concentrations of dehydroepiandrosterone and elevated blood concentration of cortisol are associated with increased atherosclerosis. For example, patients with Cushing's Disease exhibit coronary artery disease at a rate four times higher than the general population. Many conditions associated with increased cortisol secretion, such as depression, low birth weight, advancing age, hostility, and mental stress predispose to coronary disease. Other conditions known to be related to the risk of cardiovascular disease such as insulin resistance, central obesity display increased cortisol secretion and stress-related cortisol secretion was associated with visceral adiposity, blood pressure and increased concentrations of glucose and insulin. Decrease dehydroepiandrosterone concentrations are correlated with future cardiovascular disease events and extent of angiographically documented coronary disease. Dehydroepiandrosterone feeding prevents atherosclerosis in cholesterol-fed animals. The following is known from the teachings of others.

Subjects with insulin resistance have a similar metabolic profile to patients with Cushing's Disease, who are known to have increased CVD mortality and increased atherosclerosis even years after successful cure. 24-hour cortisol rhysmicity may be responsible, at least in part, for the diurnal variation in glucose tolerance. Mental stress results in elevation in cortisol, and this increase is attenuated by estrogen, which is known to prevent atherosclerosis and stress-mediated cortisol secretion was positively correlated with visceral obesity, insulin, glucose and blood pressure. Depressed patients have increased CAD incidence and have increased diurnal plasma concentration of cortisone and this correlates with increased fasting insulin and glucose and increased visceral obesity. Men have a higher rate of cortisol production than women and have a higher incidence of cardiovascular disease. Vital exhaustion and hostility increase adrenal responsiveness to adrenocortical stimulating hormones and hostility is associated with increased coronary calcification, a marker for coronary artery disease.

Glycemic control in diabetic patients deteriorates following even minor everyday stress. Normal morning rise in cortisol inhibits lipolysis and this is reversible by metyrapone. Increased age is associated with increased endogenous glucose production, increased cortisol production, and decreased dehydroepiandrosterone production. Syndromes with insulin resistance, such as polycystic ovary disease, are associated with increased adrenal sensitivity to adrenocortical stimulating hormone during insulin-induced hypoglycemia and increased secretion of cortisol.

Women with increased abdominal fat show increased and prolonged cortisol secretion following mental stress. People with low birth weight, who are known to be at increased risk of insulin resistance, have elevated plasma cortisol concentrations.

The incidence of cardiovascular disease events and the presence of narrowing of the coronary arteries by angiography are associated with decreased concentrations of dehydroepiandrosterone in men and dehydroepiandrosterone supplementation inhibits atherosclerosis in animal models. Metabolic parameters associated with insulin resistance, in particular, central obesity, are associated with evidence of increased coronary artery disease.

11-beta-hydroxysteroid dehydrogenase type 1 knockout mice (which have decreased peripheral conversion of inactive 11-dehydrocortisone to active corticosterone) are resistant to stress-induced hyperglycemia and obesity.

Patients with essential hypertension have increased urinary free cortisol (a marker of increased cortisol production). Blockage of glucocorticoid receptors with RU 486 ameliorated diabetes in OB/OB mice who display massive obesity and diabetes. Glucocorticoids inhibit Glut 4 receptor expression and insulin and noninsulin-induced trans-membrane glucose transport.

Glucocorticoids promote adipose tissue-mediated production of plasminigen activator inhibitor 1 (a protein which promotes the formation of blood clots production by human adipose tissue). Glucocorticoids inhibit the availability of tretrahydropterin and nitric oxide and inhibit flow-mediated vasodilatation, which are normally protective against atherosclerosis. In cell culture, pulse treatment with dexamethasone promotes smooth muscle proliferation, promotes cholesteryl ester formation and impairs cholesterol egress from lipoprotein depos by HDL.

### SUMMARY OF THE INVENTION

The invention relates to the use of inhibitors of adrenal synthesis for specific medical conditions. By inhibiting the secretion of glucocorticoids and promoting the secretion of dehydroepiandrosterone by the adrenal gland, this treatment will block or reverse the processes cited above leading to improved glucose tolerance, reduced blood pressure, decreased obesity, in particular, central obesity, and reduced atherosclerosis. Of particular interest, is to specifically inhibit enzyme 3-betahydroxysteroid deydrogenase. As its preferred embodiment, the present invention is directed to the use of an inhibitor comprising a member selected from the group consisting of epostane and trilostane, a specific inhibitor of 3-beta HSD via a wide variety of delivery systems.

### DETAILED DESCRIPTION OF THE INVENTION

This invention relates to the use of epostane, trilostane as well as any other inhibitor of adrenal gland synthetic pathway, delivered transcutaneously, sublingually, orally, rectally or via any other delivery route. Use of these compounds is specifically designed to reduce the abnormal secretion of cortisol in response to stress. As described in the background section, inhibition of the increased secretion of cortisol in response to stressful stimuli such as anxiety, anger, depression, fear and others, will result in amelioration of glucocorticoid- mediated insulin resistance, hypertension, obesity and atherosclerosis.

Such use for inhibitors of adrenal enzyme has not previously been proposed and is therefore unique and novel.

It will be appreciated by those skilled in the art that the application of trilostane or other inhibitors of adrenal enzyme synthesis can be used not only for treatment of an existing condition but also extends to prophylaxis. Trilostane, epostane or other enzyme inhibitors can be administered in any conventional way and the invention therefore includes within its scope pharmaceutical compositions including active ingredients and one or more physiologically acceptable diluents or carriers. The compounds according to the present invention may, for example, be formulated for oral, transcutaneous, buccal, sublingual, parenteral, local or rectal administration. Local administration includes administration by insufflation and inhalation. Examples of various types of preparation for local or transcutaneous administration include ointments, lotions, creams, gels, foams, preparations for delivery by transdermal patches, powders, sprays, aerosols, capsules or cartridges for use in a inhalator or insufflator or drops in the form, for example, of eye or nose drops, solutions and suspensions for nebulisation, suppositories, pessaries, retention enemas and chewable or suckable tablets or pellets. Active ingredients can also be contained in a liposome or microencapsulation preparation.

Ointments, creams and gels may, for example, be formulated with an aqueous or oil base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminum stearate, cetostearyl alcohol, polyethylene glycols, wool fat, bees wax, carboxypropyl methylene and cellulose derivatives and/or glyceryl monostearate and/or non-ionic emulsifying agents.

Lotions may be formulated with an aqueous or oily base and will, in general, also contain one or more emulsifying agents, stabilizing agents, dispersing agents, suspending agents or thickening agents.

Powder for external application may be formed with the aid of any suitable powder base, for example, talc, lactose or starch. Drops may be formulated with an aqueous or non-aqueous base also comprising one or more dispersing agents, solubilizing agents, suspending agents or preservatives.

Spray compositions may, for example, be formulated as aqueous solutions or suspensions or as aerosols delivered from pressurized packs, such as a metered dose inhaler with the use of a suitable liquified propellant. Aerosol compositions suitable for inhalation can be either a suspension or a solution and generally contain a composition of the active adrenal enzyme inhibitor in a suitable propellant such as a fluorocarbon or hydrogen-containing chlorofluorocarbon or mixtures thereof. The aerosol composition may optionally contain additional formulation expedience well-known to those in the arts such as surfactants, e.g. EXosurf™, a colfosceril palmitate cetyl alcohol tyloxapol, sold by Glaxo-Wellcome or lecithin and co-solvents, e.g. ethanol or other alcohols.

Capsules and cartridges for use in an inhaler or insufflator of, for example, gelatin, may be formulated containing a powder mix for inhalation of a compound of the invention and a suitable powder base such as lactose or starch. Each capsule or cartridge may generally contain between 10 to 1000 mg of the adrenal enzyme inhibitor. Alternatively, the active ingredients of the invention may be presented without expedience such as lactose.

The proportion of active ingredients and the local compositions according to the invention depends on the precise type of formulation to be prepared but generally will be within the range from 0.01 to 5.0% by weight. Generally, for most types of preparations, the preferred proportion will be within the range of from 0.1 to 2.0% by weight and preferably from 0.5 to 1.0% by weight.

Aerosol formulations are preferably arranged so that each metered dose or “puff” of aerosol contains 1 to 1000 micrograms, preferably about 10 to 200 micrograms of active ingredients. Administration may be 1 to 2000 micrograms

What is claimed is:

1. A method of inhibiting secretion of cortisol in a patient in response to stressful stimuli comprising administering an adrenal enzyme inhibitor to said patient.

2. The method of claim 1 wherein said adrenal enzyme inhibitor comprises a member selected from the group consisting of trilostane and epostane.

3. The method of claim 1 wherein said adrenal enzyme inhibitor inhibits at least one glucocorticoid.

4. The method of claim 1 wherein administering said adrenal enzyme inhibitor is carried out to promote secretion of dehydroepiandrosterone by the adrenal gland of said patient.

5. The method of claim 1 wherein said adrenal enzyme inhibitor is administered in concentrations to provide improved glucose tolerance in said patient.

6. The method of claim 1 wherein said adrenal enzyme inhibitor is administered in concentrations to provide a reduction in blood pressure in said patient.

7. The method of claim 1 wherein said adrenal enzyme inhibitor is administered in concentrations to provide a reduction in obesity in said patient.

8. The method of claim 1 wherein said adrenal enzyme inhibitor is administered in concentrations to provide a reduction in atherosclerosis in said patient.

9. The method of claim 1 wherein said adrenal enzyme inhibitor inhibits 3-beta hydroxysteroid dehydrogenase.

10. The method of claim 1 wherein said adrenal enzyme inhibitor is administered sublingually to said user.

11. The method of claim 1 wherein said adrenal enzyme inhibitor is administered orally to said user.

12. The method of claim 1 wherein said adrenal enzyme inhibitor is administered rectally to said user.

13. The method of claim 1 wherein said adrenal enzyme inhibitor is administered transcutaneously.

14. The method of claim 1 wherein said adrenal enzyme inhibitor is administered by insufflation.

15. The method of claim 1 wherein said adrenal enzyme inhibitor is administered by inhalation.

16. The method of claim 1 wherein said adrenal enzyme inhibitor is administered transdermally.

17. A method of reducing secretion of cortisol in a user subjected to stress, said method comprising administering an adrenal enzyme inhibitor in a suitable carrier to said user.

18. The method of claim 17 wherein said stress comprises stimuli selected from the group consisting of anxiety, anger, depression and fear.

19. The method of claim 17 wherein said adrenal enzyme inhibitor comprises a member selected from the group consisting of trilostane and epostane.

20. A method of improving glucose tolerance in a user comprising administering to said user, an effective amount of a composition to inhibit adrenal enzyme synthesis.

21. A method of reducing obesity of a user comprising administering to said user an effective amount of a composition to inhibit adrenal enzyme synthesis.

22. A method of reducing diabetes of user comprising administering to said user an effective amount of a composition to inhibit adrenal enzyme synthesis.

23. A method of reducing hypertension of a user comprising administering to said user, an effective amount of a composition to inhibit adrenal enzyme synthesis.

24. A method of reducing atherosclerosis of a user comprising administering to said user an effective amount of a composition to inhibit adrenal enzyme synthesis.

25. A composition for inhibiting adrenal enzyme synthesis in a user, said composition comprises an adrenal enzyme inhibitor in a suitable carrier in a concentration suitable for said inhibition.

26. The composition of claim 25 wherein said adrenal enzyme inhibitor comprises a member selected from the group consisting of trilostane and epostane.

27. The composition of claim 25 wherein at least one glucocorticoid is inhibited by said adrenal enzyme inhibitor.

28. The composition of claim 25 wherein said adrenal enzyme inhibitor is contained in said carrier in concentrations to provide improved glucose tolerance in said user.

29. The composition of claim 25 wherein said adrenal enzyme inhibitor is contained in concentrations to provide a reduction in blood pressure in said user.

30. The composition of claim 25 wherein said adrenal enzyme inhibitor is contained in concentrations to provide a reduction in obesity in said user.

31. The composition of claim 25 wherein said adrenal enzyme inhibitor is contained in concentrations to provide a reduction in atherosclerosis of said user.

32. The composition of claim 25 wherein said adrenal enzyme inhibitor inhibits 3-beta hydroxysteroid dehydrogenase.

33. The composition of claim 25 wherein said adrenal enzyme inhibitor is administered sublingually to said user.
